(19) United States
(12) Patent Application Publication
(10) Pub. No.: US 2024/0301511 A1
(43) Pub. Date: Sep. 12, 2024

(54) METHOD, APPARATUS, DEVICE AND MEDIUM FOR THE IDENTIFICATION OF CANDIDATE GENES THAT REGULATE THE SHAPE OF BACTERIA

(71) Applicant: Anhui Agricultural University, Hefei (CN)

(72) Inventors: Guomin HAN, Hefei (CN); Qi LIU, Hefei (CN); Chuangchuang XU, Hefei (CN); Shunli HU, Hefei (CN)

(21) Appl. No.: 18/220,800

(22) Filed: Jul. 11, 2023

(30) Foreign Application Priority Data
Mar. 7, 2023 (CN) 202310210018.3
Jun. 15, 2023 (CN) 202310714738.3

(51) Int. Cl. C12Q 1/689; G16B 5/00; G16B 20/00; G16B 25/10; G16B 40/20

(52) U.S. Cl. CPC C12Q 1/689; G16B 5/00; G16B 20/00; G16B 25/10; G16B 40/20

(57) ABSTRACT

The present application relates to a method, apparatus, device and medium for identifying candidate genes that regulate the shape of bacteria. The method includes: obtaining reference genome data of bacteria and performing protein domain analysis on the reference genome data of bacteria; determining the feature value dataset for each bacterium based on the structural domains of all proteins obtained from the analysis; obtaining shape information of each bacterium; training a bacterial shape prediction model based on the shape information of each bacterium and the feature value dataset, and determining the weights of each protein domain in influencing the shape of the bacterium according to the bacterial prediction model; determining candidate genes that regulate the shape of bacteria based on the weights. This method can be used to rapidly screen out the candidate genes that regulate the shape of bacteria, and establish a new method for mining biofunctional genes.

### CROSS-REFERENCE TO RELATED APPLICATIONS

The present application claims priority to Chinese Patent Application 202310210018.3, filed on Mar. 7, 2023, and Chinese Patent Application 202310714738.3, filed on Jun. 15, 2023. Chinese Patent Application 202310210018.3 and Chinese Patent Application 202310714738.3 are incorporated herein by reference.

### TECHNICAL FIELD

The present application relates to the field of high-throughput data analysis in biology, and in particular to a method, apparatus, device and medium for the identification of candidate genes that regulate the shape of bacteria.

### BACKGROUND

As a fundamental science, biology has extensive interdisciplinary and practical value. It has made significant contributions to improving human understanding of life, promoting scientific and economic development, solving human problems, and more. And the study of gene function is a very important part of biological research. Genes are important molecules that control life processes and expression of traits. The study of gene function is essential for understanding the nature of life, studying biological development and evolution, elucidating the mechanisms of disease occurrence, etc.

Methods for mining functional genes have been available since the last century. With the development of biological technology, several commonly used methods for mining functional genes have emerged, such as conventional mutagenesis screening, whole-genome association analysis, and more. For mutagenesis screening methods, since they rely on mutations generated through chemical, physical, or biological mutagenesis of a large number of organisms, the mutation points are randomly distributed, and some mutations may be ineffective or irrelevant to the research object. Thus, it requires a substantial amount of time and effort to identify and screen meaningful mutations to obtain candidate genes. In addition, mutant libraries need to be maintained by preserving and reproducing large numbers of individuals. The premise of whole-genome association analysis is to collect enough materials, such as different strains of the same species, self-crossed varieties of animals and plants, etc., at a considerable cost of resources, time, and effort, before conducting genome sequencing and resequencing. Finally, candidate key genes can be identified through whole-genome association analysis. Therefore, existing technical solutions consume a significant amount of resources and time.

### SUMMARY

Based on this, it is necessary to provide a method, apparatus, device and medium for the identification of candidate genes which regulate the shape of bacteria in response to the above technical problems.

A method for identifying candidate genes which regulate the shape of bacteria includes:
obtaining reference genome data of bacteria, and performing protein domain analysis on the reference genome data of bacteria;
determining a feature value dataset based on all protein domains obtained by analysis for each bacterium;
obtaining shape information of each bacterium;
training a bacterial shape prediction model based on the shape information of each bacterium and the feature value dataset, and determining influence weights of each protein domain on bacterial shape according to the bacterial prediction model;
determining candidate genes which regulate bacterial shape based on the determined influence weights.

A method for identifying candidate genes which regulate the phenotype of a large number of species, the above method includes:
obtaining reference genome data of a large number of species and performing protein domain analysis on the reference genome data of target species;
determining a feature value dataset based on protein domains obtained from the protein domain analysis;
obtaining phenotype information of the large number of species;
training a phenotype prediction model for the large number of species based on the phenotype information and the feature value dataset, and determining influence weights of each protein domain on the phenotype of the target species according to the phenotype prediction model;
determining the candidate genes which regulate the phenotype of the target species based on the influence weights of each protein domain.

A method of regulating a phenotype of a large number of species includes:
knocking out one or more of the candidate genes of the species obtained according to the above method, to obtain a species with altered phenotype.

An apparatus for identifying candidate genes that regulate the shape of bacteria, the above apparatus includes:
the first acquisition module for obtaining reference genome data of bacteria and performing protein domain analysis on the reference genome data of bacteria;
the first determination module for determining a feature value dataset based on all protein domains obtained from the analysis of the bacteria;
the second acquisition module for obtaining shape information of the bacteria;
the processing module for training a bacterial shape prediction model based on the shape information of the bacteria and the feature value dataset, and determining influence weights of each protein domain on the shape of the bacteria according to the bacterial prediction model;
the second determination module for determining candidate genes which regulate the shape of bacteria based on the influence weights of each protein domain.

A computer device comprising a memory, a processor, and a computer program stored in the memory and executable on the processor, wherein the processor implements the steps of the above method when executing the computer program.

A non-transitory computer-readable storage medium storing a computer program, wherein the computer program is executed by a processor to implement the steps of the above method.

The above method, apparatus, device and medium for determining candidate genes that regulate the shape of bacteria involve acquiring reference genome data of bacteria, analyzing protein domains in the reference genome data of bacteria to determine feature value data sets; obtaining shape information of each bacterium; training a bacterial shape prediction model using the shape information of each bacterium and the feature value data set, determining the impact weight of each protein domain on bacterial shape according to the bacterial prediction model; determining candidate genes that regulate bacterial shape based on the impact weights of each protein domain. This application achieves rapid preliminary screening of key genes by training a bacterial shape prediction model and determining the impact weights of each protein domain on shape during the model training process. Subsequent functional identification based on these candidate genes can greatly shorten the time for identifying bacterial gene function.

### DESCRIPTION OF THE DRAWINGS

FIG. 1 is a schematic diagram of a method for identifying candidate genes that regulate bacterial shape in an embodiment;

FIG. 2 is a precision curve of a bacterial shape prediction model in an embodiment;

FIG. 3 is a scanning electron microscope morphological observation of wild-type E. coli BL21 in an embodiment;

FIG. 4 is a graph of scanning electron microscopic morphological observations of E. coli with knockout gene MreB in an embodiment;

FIG. 5 is a graph of scanning electron microscopic morphological observations of E. coli with knockout gene Pal in an embodiment;

FIG. 6 is a schematic diagram of a method for identifying candidate genes that regulate species phenotype in an embodiment;

FIG. 7 is a structural diagram of an apparatus for the identification of candidate genes regulating the shape of bacteria in an embodiment;

FIG. 8 is an internal structure diagram of a computer device in an embodiment.

### DETAILED DESCRIPTION OF THE INVENTION

In order to make the purpose, technical solution and advantages of this application clearer, the following detailed description of this application is provided in conjunction with the accompanying drawings and embodiments. It should be understood that the specific embodiments described here are only used to explain this application and are not intended to limit this application.

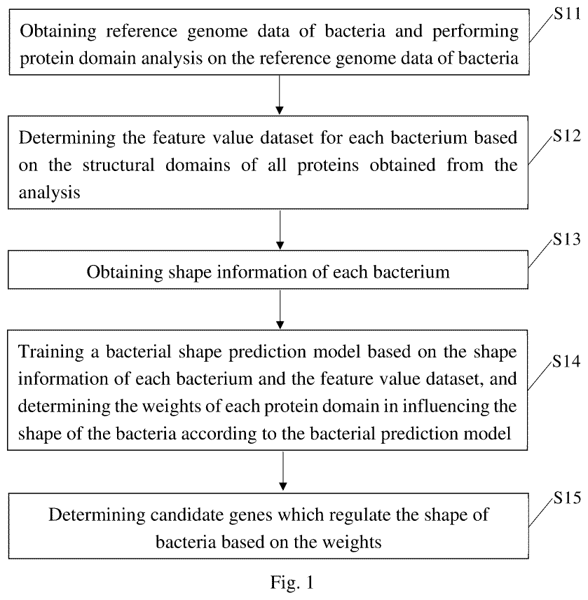

In one embodiment, as shown in FIG. 1, a method for identifying candidate genes that regulate bacterial shape is provided. The method is illustrated by applying it to a server and includes the following steps:
S11. Obtain reference genome data of bacteria and perform protein domain analysis on the reference genome data.

In this application, the server can pre-obtain the above-mentioned reference genome data of the bacteria from a pre-set database. The pre-set database can be NCBI (National Center for Biotechnology Information) database or ensembl database, etc. The above-mentioned reference genome data of the bacteria refers to data related to the genes of the bacteria, e.g., base sequences of bacteria, encoded protein sequences, etc.

Furthermore, the server performs protein domain analysis on the reference genome data of the bacteria, specifically extracting corresponding protein domains from the protein sequences. In this application, pfam_scan software can be used to analyze the above-mentioned reference genome data of the bacteria.
S12. Based on the protein domain structures of each bacterium obtained after analysis, the feature value dataset was determined.

In this application, the aforementioned protein domain is a functional structural unit that constitutes a protein, and is used to represent the different functions and features that the protein possesses. Protein domains are composed of amino acid sequences and assembled together in three-dimensional space to form “domain structures”. The function and structure of a protein are mainly determined by their domain structures. Therefore, this application selects the protein domain structures of bacteria as features for model training.

Furthermore, this application can analyze the protein domain structures of bacteria that have already been analyzed, conduct frequency statistics on the protein domain structures obtained after analysis, construct a protein domain frequency matrix based on the statistical results, and obtain a feature value dataset containing multiple bacterial protein domain structures.

S13. Obtain the shape information of each bacterium.

In this application, the aforementioned shape information refers to the information that describes the external morphology of the bacterium from aspects such as length, width, and other external features. For example, the shape of the bacteria mentioned above can include rod-shaped, spherical, spiral, and others such as star-shaped, filamentous, brick-shaped, pleomorphic, circular, etc.

Specifically, this application can obtain the aforementioned shape information by querying a bacterial information database. For example, the Bacterial Information Query Website BacDive can be used to query the known shape information of bacterial species, totaling 4847 kinds (as of June 2022), of which there are 3991 kinds of rod-shaped bacteria, followed by 460 kinds of spherical bacteria, and 79 kinds of spiral bacteria. Other shapes such as star-shaped, filamentous, brick-shaped, pleomorphic, circular, etc. have 317 species.

S14. Train a bacterium shape prediction model based on the shape information and the feature value dataset of each bacterium, and determine the influence weights of each protein domain on the bacterial shape according to the bacterial prediction model.

In this application, because the classification of the shape information of bacteria obtained directly from the database is too messy to predict one by one, in order to facilitate the construction of the subsequent model, the shape information of bacteria is divided into four categories according to classical classification methods: coccus, rod, spiral, and other bacteria. Each bacterium is classified into one of these four categories. Therefore, the shape information of bacteria in this application can include spherical, rod-shaped, spiral, and other.

In this application, the Random Forest algorithm can be used to train the model. The Random Forest algorithm is a machine learning method based on decision trees. It completes classification or regression tasks by constructing multiple decision trees and voting on them. The Random Forest algorithm can effectively overcome the problem of overfitting of a single decision tree and has good performance in various machine learning tasks.

Specifically, the steps of the Random Forest algorithm can include:
Randomly select a subset of the training data with replacement as a random subset; For each random subset, construct different decision tree models for training;
Finally, combine the constructed multiple decision trees into a forest and use the constructed Random Forest for prediction or classification.

Here, the aforementioned influence weight refers to the degree of influence or decisiveness of each protein domain on the shape of the bacterium. In the Random Forest algorithm in this application, the “importance” function can be used to evaluate the degree of influence of each protein domain on the shape of the bacteria, where the higher the influence weight ranking, the higher the decisiveness of the protein domain on the shape of the bacteria.
S15. Determine candidate genes that regulate bacterial shape based on the respective influence weights.

In this application, the server can determine the number M of key features based on the rankings of the respective influence weights and the prediction error rate of the aforementioned bacterial shape prediction model. The key features refer to the protein domains mentioned above. The server further obtains the top M protein domains corresponding to the influence weights as key features, and obtains the genes corresponding to these M key features from a pre-set database to obtain candidate genes that regulate bacterial shape.

In one embodiment, the protein domain frequency matrix is constructed based on all protein domains of all bacteria obtained after parsing to form a feature value dataset, may include:

Based on the analyzed protein domains of all bacteria, a protein domain frequency matrix is constructed to obtain the feature value dataset.

In this application, the process of constructing the protein domain frequency matrix based on all protein domains of all bacteria obtained after parsing may include:
Parsing the genes of each bacterium to obtain its protein domains; Concatenating the protein domains of each gene to form column names; Counting the frequency of each domain to construct the protein domain frequency matrix.

In this application, each bacterium includes multiple genes, and each gene may generate one or more protein domains. The multiple protein domains obtained from parsing may be the same or different. When multiple protein domains are obtained from a gene, they need to be concatenated to obtain a combination of protein domains. The concatenated combination of protein domains is used as a feature to construct the aforementioned protein domain frequency matrix.

Furthermore, the protein domain frequency matrix is constructed by counting all protein domains or combinations of each gene in each bacterium. Specifically, during counting, if a protein domain or combination appears in the current gene of the current bacterium, it is counted once. Similarly, the counting is performed to obtain the above-mentioned protein domain frequency matrix.

In this application, the obtained protein domain frequency matrix for each bacterium is the above-mentioned feature value dataset. The feature value dataset may include the names and occurrence times of each protein domain for each bacterium.

Please refer to Table 1 below for the data structure of the feature value dataset in one embodiment of the present application.

TABLE 1

| | bacterium | bacterium | bacterium | bacterium |
|---|---|---|---|---|
| | 1 | 2 | 3 | 4 |
| Protein domain A | 1 | 5 | 0 | 2 |
| The combination of | 5 | 10 | 1 | 4 |
| protein domain B | | | | |
| and domain A. | | | | |
| The combination of | 0 | 1 | 9 | 1 |
| multiple protein | | | | |
| domains C | | | | |
| Protein domain D | 8 | 1 | 1 | 1 |

As shown in Table 1 above, the feature value dataset may include the names of each protein domain corresponding to each gene of each bacterium, as well as the occurrence times of each protein domain or combination in each bacterium.

It should be noted that in this application, after parsing the protein domains of each bacterium, it may include single protein domains or multiple protein domains. Furthermore, multiple protein domains can be concatenated to obtain a combination of protein domains.

For example, when parsing the genes of the current bacterium, if protein domain B and protein domain A are obtained, protein domain B and protein domain A are further concatenated in order, and the concatenated combination is used as a feature in the feature value dataset.

Similarly, if three protein domains C are obtained when parsing the genes of the current bacterium, the three protein domains C are further concatenated, and the concatenated combination is used as a feature in the feature value dataset.

Through this embodiment in this application, all protein domains of each bacterium can be obtained as features for model training, so that the trained model can reflect the degree to which protein domains affect shape, and then candidate genes determining the shape of bacteria can be identified through this degree of influence.

In one embodiment, training a bacteria shape prediction model based on the shape information of each bacterium and the feature value dataset may include:
Determining a grouping list based on the shape information of each bacterium, including a test group and a training group;
Performing multiple rounds of training using the shape information and feature value dataset of each bacterium in the training group;
Obtaining prediction indicator values for the test group predicted by the models obtained from each round of training, adjusting the proportion of bacterial species corresponding to each shape in both the test and training groups based on the prediction indicator values obtained, and updating the grouping list for the next round of training;
When the prediction indicator value reaches a predetermined threshold, the obtained model is the bacterial shape prediction model.

In this application, the grouping list mentioned above refers to a list that includes two groups: a training set and a test set. The training set is equivalent to the aforementioned training group, and the test set is equivalent to the aforementioned test group. In this application, the model is trained using the training set, and the models obtained from each round of training are used to predict the test set to verify the quality of the model.

In this application, in determining the grouping list based on the shape information of each bacterium, it can specifically include:
Classifying each bacterium according to its shape information, this application can classify all bacteria into four categories, namely spherical, rod-shaped, spiral, and other shapes. That is, after classification, the shape of each bacterium falls under one of the four major categories mentioned above;
Grouping the classified bacteria into two groups: a training group and a test group.

Specifically, this application can divide all bacteria into two groups. In one possible design, the bacteria can be divided into a training group and a testing group in a 3:1 ratio, namely 75% for the training group and 25% for the testing group. It should be noted that when grouping, it is necessary to try to make the four shapes mentioned above, rod-shaped, spherical, spiral, and other shapes, covered in the training group after grouping. The same four shapes, rod-shaped, spherical, spiral, and other shapes, should also be covered in the testing group after grouping.

Please refer to Table 2 for an example of a grouping list in one embodiment.

TABLE 2

| Sample ID | The shape of bacteria | Group |
|---|---|---|
| ID1 | rod-shaped | Training group, test group |
| ID2 | spherical | Training group, test group |
| ID3 | spiral | Training group, test group |
| ID4 | other | Training group, test group |

As shown in Table 2 above, the grouping list mentioned can include two groups, with the ID of each sample included in each group as well as the corresponding shape information. It can be understood that this application has divided the above-mentioned bacteria into training and test groups, and other groups also carry the shape information and ID of each bacterium. The grouping and the aforementioned feature value dataset are used for model training to obtain the aforementioned bacterial shape prediction model.

Furthermore, the aforementioned prediction indicator refers to the ability to measure how well the trained model classifies. The prediction indicators can include accuracy, recall, and kappa coefficient. These three indicators are all calculated and analyzed based on the confusion matrix generated by prediction. Accuracy refers to the proportion of correctly predicted samples among all samples; recall, also known as sensitivity, is the probability that a positive sample is predicted to be positive among all actual positive samples. The kappa coefficient is a statistical method used to evaluate the model's classification error rate. Its value ranges from 0 to 1, and the higher the value, the higher the accuracy of the model's classification. Generally, the values of the kappa coefficient are divided into five categories, representing different levels of consistency: 0.0 to 0.20, extremely low consistency; 0.21 to 0.40, general consistency; 0.41 to 0.60, moderate consistency; 0.61 to 0.80, high consistency; 0.81 to 1, almost perfect consistency. This application continuously adjusts and optimizes the models obtained from each training session based on the above indicators to achieve better classification results and accuracy.

Specifically, the training group includes four types of shapes, rod-shaped, spherical, spiral, and other shapes, as well as the number of bacteria corresponding to each shape. Similarly, the testing group includes the same four types of shapes, rod-shaped, spherical, spiral, and other shapes, as well as the number of bacteria corresponding to each shape. When calculating the values of various indicators of the current model, if a certain indicator value is lower than the preset threshold, the training and testing groups can be adjusted by changing the proportion of bacteria of each shape, then retrained and so on until all indicators of the model reach the corresponding threshold value. The obtained model is the optimal model, which can be used as the bacterial shape prediction model mentioned above.

In a possible application scenario, in the training of a model (Model 1) for a certain session, the number of bacteria of each type in the training set are: 345 types of cocci, 2994 types of bacilli, 60 types of spiral bacteria, and 237 types of other bacteria. In the test set, the number of bacteria of each type are: 115 types of cocci, 997 types of bacilli, 19 types of spiral bacteria, and 80 types of other bacteria.

Five machine learning algorithms, namely Support Vector Machine, Conditional Inference Tree, Decision Tree, Naive Bayes, and Random Forest, were used to train models on the training set described above. From the perspective of accuracy, the Support Vector Machine algorithm had the highest error rate at 90.04%, followed by the Conditional Inference Tree, Decision Tree and Random Forest algorithms. Naive Bayes algorithm had the lowest accuracy of only 1.87%. From the perspective of recall rate, Support Vector Machine, Conditional Inference Tree, Decision Tree, and Random Forest algorithms performed well in recalling rod-shaped bacteria, with a recall rate of over 99%. However, the recall rates for spherical bacteria, spiral bacteria, and other bacteria were poor. Although Naive Bayes algorithm achieved a perfect recall rate of 100% for spiral bacteria, its performance was poor for the other three types of bacteria. From the Kappa coefficient, the performance of these five algorithms was not ideal, with the highest Kappa coefficient being 0.59 for the Support Vector Machine algorithm, indicating moderate consistency.

The models trained by each algorithm were applied to the test set for prediction. In terms of accuracy, the Random Forest algorithm had the highest prediction accuracy at 86.86%, followed by Support Vector Machine, Conditional Inference Tree, and Decision Tree algorithms, all of which had prediction accuracies above 80%. The Naive Bayes algorithm had the worst prediction accuracy at only 1.73%. Regarding recall rate, the performance of these algorithms varied greatly. The recall rate for rod-shaped bacteria was relatively good, while the recall rates for the other three types of bacteria were poor. Although Naive Bayes algorithm achieved a perfect recall rate of 100% for spiral bacteria, its recall rates for spherical bacteria, rod-shaped bacteria and other bacteria were very low. From the Kappa coefficient, Model 1 had a high misclassification rate for these five algorithms, with the Kappa coefficient being less than 0.5. Naive Bayes and Conditional Inference Tree algorithms had a Kappa coefficient of 0, indicating very low consistency in classifying Model 1.

Due to poor performance of Model 1, according to the prediction results of Model 1, it was determined that the high weight of rod-shaped bacteria in the total number of bacteria was related to the poor performance. Therefore, the proportion was adjusted by reducing the number of rod-shaped bacteria and increasing the number of spherical bacteria and spiral bacteria. Specifically, 251 strains of spherical bacteria and 160 strains of spiral bacteria were added. After adjustment, the numbers of different types of bacteria in the training set were: 534 strains of spherical bacteria, 1035 strains of rod-shaped bacteria, 178 strains of spiral bacteria, and 239 strains of other bacteria. The numbers of different types of bacteria in the test set were: 177 strains of spherical bacteria, 345 strains of rod-shaped bacteria, 60 strains of spiral bacteria, and 79 strains of other bacteria. The adjusted training set was used for the next round of model training, and so on, until the optimal model was obtained.

In a possible application scenario, after multiple rounds of training, it was found that when the parameters in Table 3 were used for grouping, the obtained model was the optimal model described above.

TABLE 3

| Bacterial type | Training group | Test group |
|---|---|---|
| spherical | 854 | 284 |
| rod-shaped | 999 | 331 |
| spiral | 589 | 196 |
| other | 373 | 124 |

In Table 3, the numbers of strains corresponding to spherical, rod-shaped, spiral, and other bacteria in the training set were 854, 999, 589, and 373 respectively. The numbers of strains corresponding to spherical, rod-shaped, spiral, and other bacteria in the test set were 284, 331, 196, and 124 respectively. The model trained with this set of parameters was the optimal model, with an accuracy of 87.89%, a recall rate of 86.18% for spherical bacteria, a recall rate of 94.49% for rod-shaped bacteria, a recall rate of 90.66% for spiral bacteria, and a recall rate of 70.51% for other bacteria. The Kappa coefficient was 0.83, indicating almost complete consistency. The prediction accuracy for the test set was 94.76%, the Kappa coefficient was 0.93, and the recall rates for spherical, rod-shaped, spiral, and other bacteria were 97.18%, 92.75%, 98.98%, and 87.90% respectively.

Through this implementation, the model parameters (the ratio of the number of different shaped bacteria in the training and test sets) can be continuously adjusted to obtain the optimal model and achieve the best prediction accuracy for bacterial shape.

In one embodiment, the weights of protein domains on bacterial shape determined by the bacterial prediction model can include:
Obtaining the decision trees in the bacterial prediction model, with each protein domain as a classification node when performing feature classification;
Obtaining the extent to which purity is reduced for the current node during the splitting process in each decision tree;
Determining the weight of the corresponding protein domain on bacterial shape based on the degree to which purity is reduced for the current node during the splitting process in each decision tree.

In this application, the random forest algorithm was used to train the bacterial shape prediction model mentioned above. Furthermore, the “importance” function in the random forest algorithm was used to evaluate the weights of different protein domains on bacterial shape.

Specifically, the random forest algorithm constructs multiple decision trees and makes decisions based on the majority vote for classification or regression tasks. The algorithm works by: sampling a subset of training data with replacement to create a random subset; building different decision tree models for each random subset; combining the constructed decision trees into a forest; using the resulting random forest for prediction or classification.

In this application, the decision tree is a machine learning method that represents a mapping relationship between object attributes and object values. The classification model of the decision tree is a tree-like structure, where each node represents an object (in this application, each node in the tree represents a protein domain), each branching path represents a possible attribute value, and each leaf node corresponds to the value of the object represented by the path traversed from the root node to the leaf node.

The generation process of a decision tree can be divided into three parts: selecting a feature from numerous features in the training data as the split criterion for the current node; recursively generating child nodes from top to bottom based on the selected feature evaluation criterion, until the data set is no longer divisible and the decision tree stops growing; finally, all nodes and edges are combined into a tree structure diagram, and the output types of all leaf nodes are classified.

Based on the principles of the random forest algorithm described above, in each training session, this application constructs multiple decision trees, where each node in the decision tree represents a protein domain. After obtaining the optimal model, i.e., the bacterial shape prediction model described above, the importance function in the random forest algorithm is used to evaluate the importance of the classification features, i.e., to evaluate the influence weights of each protein domain on bacterial shape.

Specifically, by observing the performance of each decision tree in the random forest and the features used, this application can calculate the influence weight of each feature (equivalent to a protein domain).

In one possible implementation, the reduction in purity of the current node when the current node is split in each decision tree can determine the influence weight of the corresponding protein domain on bacterial shape, may include:

The influence weight of the protein domain corresponding to the current node on bacterial shape can be calculated by obtaining the average or sum of the reduction in purity of the current node in each decision tree, that is, the degree to which the node's purity is reduced when the current node is split in each decision tree.

In one example implementation, the candidate genes that regulate bacterial shape can be determined based on the above influence weights. This can include:
Performing cross-validation on the bacterial shape prediction model to determine the relationship between the number of protein domains and the error rate of the bacterial shape prediction model;
Determining the number of key protein domains based on the relationship between the number of protein domains and the error rate of the bacterial shape prediction model;
Using the number of key protein domains and the influence weights to determine the candidate genes.

In this application, the candidate genes mentioned above refer to genes that may have a significant impact on bacterial shape determination. The key protein domains mentioned above refer to protein domains that have a significant impact on bacterial shape determination. The relationship between the number of protein domains and the error rate of the bacterial shape prediction model can be represented by the curve of the error rate of the corresponding bacterial shape prediction model under different numbers of protein domains.

In this application, the rfcv function in the randomForest package is used to perform five-fold cross-validation on the training model to obtain a model accuracy curve, which represents the relationship between the number of protein domains mentioned above and the error rate of the bacterial shape prediction model.

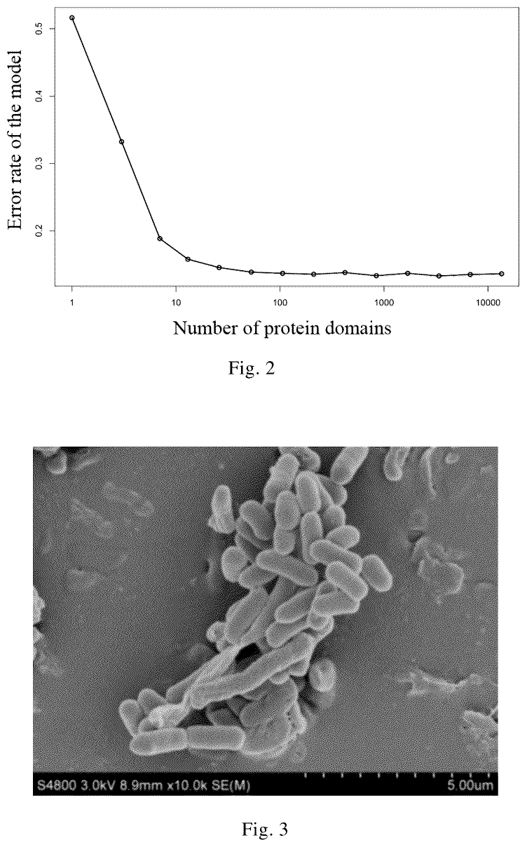

Please refer to FIG. 2, which shows an example of the accuracy curve of a bacterial shape prediction model. In FIG. 2, the horizontal axis of the model accuracy curve represents the number of protein domains (equivalent to the number of protein domains mentioned above), and the vertical axis represents the error rate of the model (equivalent to the error rate predicted by the bacterial shape prediction model mentioned above). As shown in FIG. 2, with an increase in the number of features to about 10, the error rate of the model decreases significantly. However, after the number of features exceeds 10, the curve becomes flat, indicating that the importance of subsequent features is not high and has little research value for improving the accuracy of the model. Therefore, it can be determined that the number of key protein domains is 10.

Based on the number of key protein domains and their corresponding influence weights, candidate genes that regulate bacterial shape can be identified through the following steps:
Sort the influence weights in descending order;
Obtain the top 10 influence weights and their corresponding protein domains from the sorted list;
Obtain the genes corresponding to the protein domains to obtain the candidate genes mentioned above.

Please refer to Table 4, which shows an example of the sorted list of influence weights mentioned above.

TABLE 4

| | | | | Influence | | | |
|---|---|---|---|---|---|---|---|
| | Influence | Influence | Influence | weights | Average | Average | |
| | weights of | weights of | weights of | of other | accuracy | impurity | |
| Domain | cocci | bacilli | spirilla | shapes | decline | reduction | Gene |
| OmpA | 0.005285 | 0.002413 | 0.005321 | 0.00515 | 0.004262 | 6.238621 | Pal |
| YicC_N | 0.001397 | 0.002047 | 0.001862 | 0.003572 | 0.002016 | 1.886309 | yicC |
| FlaA | 0.000973 | 0.001771 | 0.011123 | 0.001188 | 0.003407 | 5.90406 | — |
| MreB_Mb1 | 0.007044 | 0.001759 | 0.00416 | 0.003285 | 0.004038 | 4.691432 | MreB |
| MotA_ExbB | 0.007018 | 0.001662 | 0.004844 | 0.004291 | 0.004293 | 5.899423 | tolQ |
| FtsX_ECD | 0.000809 | 0.001579 | 0.004971 | 0.003328 | 0.002283 | 2.331755 | FtsX |
| Amidase_3 | 0.000697 | 0.001513 | 0.000851 | 0.001527 | 0.00113 | 1.499186 | amiC |
| Plug | 0.001401 | 0.001462 | 0.001479 | 0.0014 | 0.001435 | 1.521337 | yddB |
| FlgD | 0.003965 | 0.001427 | 0.005393 | 0.004115 | 0.003401 | 3.466897 | — |
| RNA_pol_Rpb6 | 0.000605 | 0.001347 | 0.005913 | 0.001938 | 0.002155 | 4.183334 | rpoZ |

As shown in Table 4, the top ten influence weights of each domain(s) were selected and sorted in descending order according to the influence weights of protein domains in bacilli. The genes corresponding to the top ten protein domains were identified as candidate genes regulating bacterial shape, and used for subsequent gene knockout validation. From Table 4, it can be seen that Pal is the gene with the highest weight for affecting the shape of rod-shaped bacteria. When using E. coli as the target bacterium for gene knockout verification, if the shape of E. coli changes from rod-shaped to other shapes after knocking out the Pal gene, then Pal is considered to be the key gene that regulates the rod shape of E. coli.

Through the implementation of this application, it is possible to obtain the influence weights of protein domains on bacterial shape based on the trained bacterial shape prediction model. At the same time, the number of key protein domains can be determined based on the accuracy curve of the model, thus accurately obtaining the candidate genes mentioned above.

In one embodiment, the method further includes:
Obtaining the shape information of the target bacteria after knocking out the candidate genes;
If the shape information of the target bacteria after knocking out the candidate genes is the same as that before knockout, it can be determined that the candidate gene is not a key gene regulating the shape of the target bacteria;
If the shape information of the target bacteria after knocking out the candidate genes is different from that before knockout, it can be determined that the candidate gene is a key gene regulating the shape of the target bacteria.

In one embodiment, the method further includes:
Knocking out the candidate genes of the target bacteria and culturing the target bacteria with the knocked-out candidate genes.

In this application, the target bacteria mentioned above refer to the bacteria selected for experiments to verify whether the candidate genes can regulate their shape. For example, E. coli, which has a rod-shaped morphology, can be selected as the target bacterium. In the experimental verification, the candidate genes in E. coli can be knocked out, and the shape of the bacterium after knockout can be observed to determine whether the candidate gene is a key gene regulating the rod shape of the target bacterium.

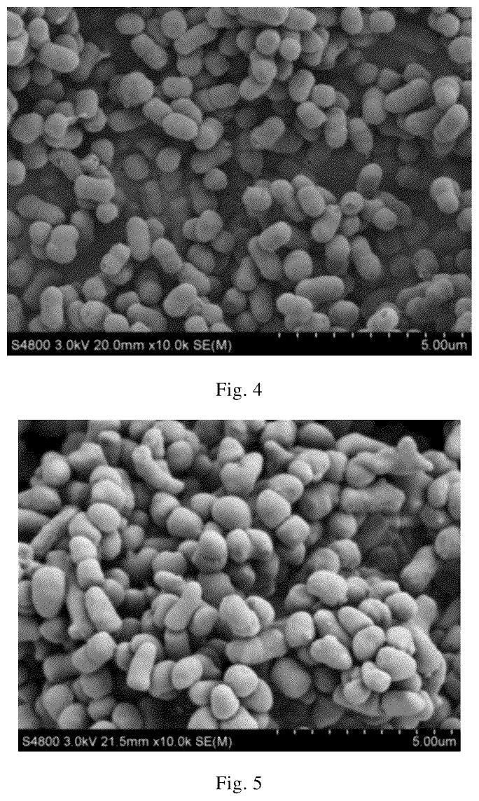

In this application, the growth shape mentioned above refers to the shape of the bacteria grown after knocking out the candidate genes. Taking E. coli as an example of rod-shaped bacteria and the candidate gene MreB as an example to verify this method, wild-type E. coli BL21 shown in FIG. 3 has a long or short rod-shaped morphology with rounded ends. The electron microscopy results showed that the E. coli with MreB gene knocked out presented a shape similar to a sphere, with a significantly reduced length but no significant change in diameter (FIG. 4). Therefore, after knocking out the candidate gene MreB, the shape of E. coli changed from a rod shape to a spherical shape, which verified that the gene MreB was a key gene regulating the rod shape of the bacterium.

Taking another candidate gene Pal as an example to verify this method, wild-type E. coli BL21 shown in FIG. 3 has a typical rod-shaped morphology according to electron microscopy results. However, after knocking out the Pal gene, the length of E. coli became shorter, the diameter increased, and the shape became irregular, resembling a protoplast without a cell wall (FIG. 5). Therefore, it was verified that the Pal gene was also a key gene regulating the rod shape of the bacterium.

Furthermore, although the length of E. coli with yicC, tolQ, amiC, yddB, and rpoZ genes knocked out did not change significantly, the surface produced many folds and indentations, indicating that these five genes affect the surface morphology of the bacterium. E. coli with the ftsX gene knocked out remained smooth and rounded in shape, with no significant difference from the wild type, verifying that the ftsX gene was not a key gene regulating the shape of the bacterium.

This application can quickly identify key genes regulating the shape of bacteria from candidate genes through the disclosed method.

In one embodiment, the method may also include the following steps:
When the number of candidate genes that are not key genes regulating the shape of the target bacteria exceeds a preset number, the step of determining the number of key protein domains based on the relationship between the number of protein domains and the error rate of the bacterial shape prediction model determined by it;
Determining new candidate genes based on the newly determined number of key protein domains and their respective influence weights;
Returning to the step of obtaining information about the shape of the target bacteria after knocking out the candidate genes with the new candidate genes.

In this application, when the number of candidate genes that are not key genes regulating the shape of the target bacteria exceeds a preset number, it indicates that most of the selected candidate genes are not the key genes that need to be found. In consideration of possible omissions, a new number of key protein domains can be obtained. The preset number mentioned above can be set according to actual needs, and the new number is greater than the original number.

Specifically, this application can determine the target interval of the number of protein domains based on the relationship between the number of protein domains and the error rate of the bacterial shape prediction model;
It obtains the new number of key protein domains from the target interval.

Among them, the number of protein domains in the target interval satisfies the following conditions:
With the increase of the number of protein domains, the change value of the accuracy of the bacterial shape prediction model is lower than the preset threshold.

For example, if the original number is ten and the target interval determined is [10, 15], the new number of key protein domains, such as 12, can be obtained from this interval. Based on the obtained new number of key protein domains, the top 12 protein domains ranked by influence weight are obtained. Then, new candidate genes are determined based on the 12 protein domains.

In one embodiment, the above-mentioned method may further include:

When the number of times of executing the step of determining the new number of key protein domains exceeds a preset number, the process ends.

In this application, if there still are more candidate genes that are not key genes regulating the shape of the target bacteria than the preset number after multiple cycles (exceeding the preset number of times), the loop is terminated and the process ends.

By adopting this embodiment, this application can avoid omissions by obtaining a new batch of candidate genes.

In this application, the candidate gene identification method provided in the above embodiment is used to identify candidate genes regulating bacterial shape. However, considering other species such as animals, plants, and fungi, etc., there is also a need for rapid mining of gene sequences. Therefore, similar candidate gene identification methods as provided in the above embodiment can be used to identify candidate genes for other species.

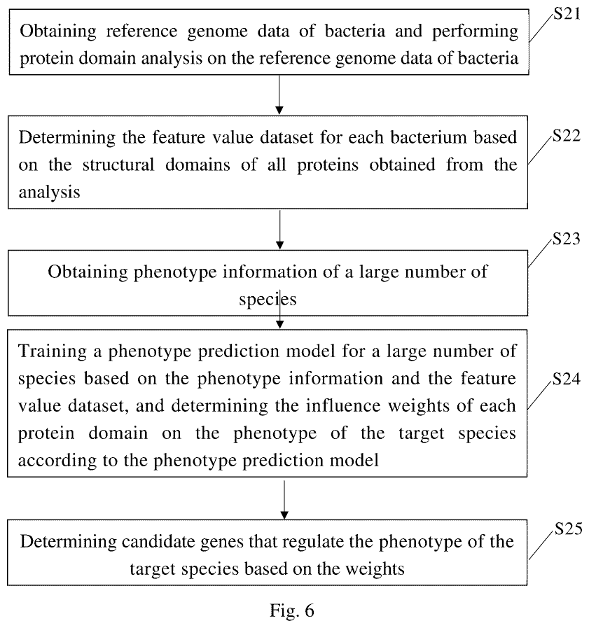

Specifically, please refer to FIG. 6. This application also provides a method for identifying candidate genes that regulate the phenotype of a large number of species, which may include:
S21. Obtaining reference genome data of a large number of species and performing protein domain analysis on the reference genome data of the target species;
S22. Determining a feature value dataset based on the protein domains obtained from the analysis;
S23. Obtaining phenotype information of a large number of species;
S24. Training a phenotype prediction model for a large number of species based on the phenotype information and the feature value dataset, and determining the influence weights of each protein domain on the phenotype of the target species according to the phenotype prediction model;
S25. Identifying candidate genes that regulate the phenotype of the target species based on the influence weights of each protein domain.

In this application, the target species referred to above refers to various species that require phenotype prediction. The target species here includes bacteria as described in the above embodiment, as well as other species such as animals, plants, fungi, etc. The phenotype information mentioned above refers to the individual morphology, function, and other aspects of the target species to be investigated. This phenotype information includes the shape information mentioned in the above embodiment and can also include other characteristics such as height, color, etc. The phenotype prediction model mentioned above refers to a model that can predict the phenotype of the target species. Here, the phenotype prediction model can include the bacterial shape prediction model as described in the above embodiment, and can also include accurate prediction models obtained using other algorithms of machine learning and deep learning. The influence weights mentioned above refer to the decisive size of each protein domain on the phenotype of the target species. The candidate gene mentioned above refers to genes that may regulate the phenotype of the target species, and experimental identification based on these candidate genes can be performed on the key genes that affect the phenotype of the target species.

The specific implementation of steps S21-S25 in this application is fundamentally the same as that of steps S11-S15. Therefore, the specific limitations of steps S21-S25 can refer to the specific limitations of steps S11-S15 mentioned above, and will not be repeated here.

The method for identifying candidate key genes that regulate bacterial shape based on genomic information in this invention saves a lot of experimental research costs and time, creating a new method for mining functional genes in biology. In the same inventive concept as this method, this application also provides a method for identifying candidate genes that regulate the phenotype of different species, which can be used for other features and other types of species. By using multiple different species' genomic sequences and known phenotypes or metabolite values, this new method can quickly determine the unique key gene composition of a certain type of species, which is different from the method of mining functional genes for a single species.

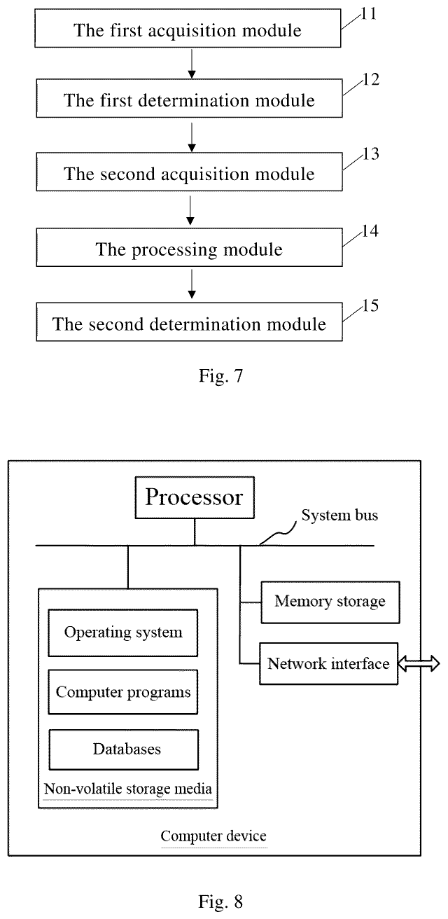

In one embodiment, as shown in FIG. 7, an apparatus for identifying candidate genes that regulate bacterial shape is provided. The apparatus includes the first acquisition module 11, the first determination module 12, the second acquisition module 13, the processing module 14, and the second determination module 15. Specifically:
The first acquisition module 11 is used to obtain reference genome data of the bacteria and perform protein domain analysis on the reference genome data;
The first determination module 12 uses all protein domains obtained from the analysis to determine the feature value dataset;
The second acquisition module 13 is used to obtain shape information of each bacterium;
The processing module 14 is used to train a bacterial shape prediction model based on the shape information of each bacterium and the feature value dataset, and to determine the influence weights of each protein domain on bacterial shape using the bacterial prediction model;

The second determination module 15 is used to identify candidate genes that regulate bacterial shape based on the influence weights determined by the processing module.

The specific limitations of the apparatus for identifying candidate genes that regulate bacterial shape can be referred to the limitations of the method for identifying candidate genes that regulate bacterial shape described above and will not be repeated here. The various modules in the above-mentioned device for identifying candidate genes that regulate bacterial shape can be implemented wholly or partially by software, hardware, and their combinations. These modules can be embedded in or independent of the processor in the form of hardware, or stored in the memory of the computer device in the form of software, enabling the processor to call and execute the corresponding operations of each module.

In one embodiment, a computer device is provided, which can be a terminal or a server. The internal structure of the computer device can be as shown in FIG. 8, including a processor, memory, and network interface connected through a system bus. The processor of the computer device is used to provide computing and control capabilities. The memory of the computer device includes non-volatile storage media and main memory. The non-volatile storage media stores the operating system and computer programs. The main memory provides an environment for the operation of the operating system and computer programs stored in the non-volatile storage media. The network interface of the computer device is used to communicate with external terminals via a network connection. When the computer program is executed by the processor, it realizes a method for identifying candidate genes that regulate bacterial shape.

In one embodiment, a computer device is provided, which includes memory, processor, and a computer program stored in the memory and runnable on the processor. When the processor executes the computer program, it implements the steps of the method for identifying candidate genes that regulate bacterial shape described in any of the above embodiments.

In another embodiment, a computer-readable storage medium is provided, which stores a computer program. When the computer program is executed by the processor, it implements the steps of the method for identifying candidate genes that regulate bacterial shape described in any of the above embodiments.

Ordinary skilled technicians in this field can understand all or part of the processes involved in implementing the methods described above, which can be achieved by instructing related hardware through computer programs. The computer program can be stored in a non-volatile computer-readable storage medium and, when executed, includes the processes of the embodiments described method above. In the embodiments provided by this application, any reference to memory, storage, database or other media may include volatile and/or non-volatile memory. Non-volatile memory can include read-only memory (ROM), programmable ROM (PROM), erasable programmable ROM (EPROM), electrically erasable programmable ROM (EEPROM), or flash memory. Volatile memory can include random-access memory (RAM) or external high-speed cache memory. By way of illustration and not limitation, RAM may take many forms, such as static RAM (SRAM), dynamic RAM (DRAM), synchronous DRAM (SDRAM), double data rate SDRAM (DDRSDRAM), enhanced SDRAM (ESDRAM), synchronous link (Synchlink) DRAM (SLDRAM), Rambus direct RAM (RDRAM), direct RDRAM, and RDRAM bus dynamic RAM.

The various technical features of the above embodiments can be combined arbitrarily. To keep the description concise, not all possible combinations of technical features in the above embodiments are described. However, as long as the combination of these technical features does not contradict each other, it should be considered within the scope of this specification.

The above-described embodiments only express several implementation methods of the present application, which are relatively specific and detailed in description. However, this should not be understood as limiting the scope of the invention patent. It should be pointed out that ordinary skilled technicians in this field can make various modifications and improvements without departing from the conception of the present application, and these are all within the scope of protection of the present application. Therefore, the scope of protection of this patent application should be based on the appended claims.

1. A method of identifying candidate genes, which regulate shape of bacteria, comprising:
obtaining reference genome data of bacteria, and performing protein domain analysis on the reference genome data of bacteria;
determining a feature value dataset based on all protein domains obtained by analysis for each bacterium;
obtaining shape information of each bacterium;
training a bacterial shape prediction model based on the shape information of each bacterium and the feature value dataset, and determining influence weights of each protein domain on bacterial shape according to the bacterial shape prediction model; and
determining candidate genes which regulate the shape of bacteria based on the influence weights.

2. The method according to claim 1, wherein the step of determining the feature value dataset based on all protein domains obtained by the analysis for each bacterium comprises:
constructing a protein domain frequency matrix based on all protein domains obtained by the analysis for each bacterium, and obtaining the feature value dataset.

3. The method according to claim 1, wherein the step of training the bacterial shape prediction model based on the shape information of each bacterium and the feature value dataset comprises:
determining a grouping list based on the shape information of each bacterium, wherein the grouping list comprises a test group and a training group;
performing multiple trainings using the shape information of each bacterium in the training group and the feature value dataset;
obtaining prediction indicator values for the test group predicted by models trained in each round, adjusting a proportion of bacterial species corresponding to various shapes in the test group and the training group based on the prediction indicator values to obtain an adjusted grouping list, and performing next training based on the adjusted grouping list; and
obtaining the bacterial shape prediction model in response to the prediction indicator value reaching a preset threshold.

4. The method according to claim 1, wherein the step of determining the influence weights of each protein domain on bacterial shape based on the bacterial shape prediction model comprises:
obtaining each decision tree in the bacterial shape prediction model, with each protein domain used as a classification node when performing feature classification;
obtaining a degree of purity reduction of current node when performing splitting in each of the decision trees; and
determining the influence weight of a protein domain corresponding to the current node on bacterial shape based on the degree of purity reduction of the current node when performing splitting in each of the decision trees.

5. The method according to claim 1, wherein the step of determining candidate genes which regulate the shape of bacteria, based on the influence weights comprises:
performing cross-validation on the bacterial shape prediction model to determine a relationship between a number of protein domains and an error rate of the bacterial shape prediction model;
determining a number of key protein domains based on the relationship between the number of protein domains and the error rate of the bacterial shape prediction model; and
determining the candidate genes based on the number of key protein domains and the respective influence weights.

6. The method according to claim 5, wherein the method further comprises:
obtaining shape information of a target bacterium after knocking out the candidate genes;
determining that the candidate genes are not key genes for regulating a shape of target bacteria in response to the shape information after knocking out the candidate genes being identical to before; and
determining that the candidate genes are key genes for regulating the shape of the target bacteria in response to the shape information after knocking out the candidate genes being different from before.

7. The method according to claim 6, wherein the method further comprises:
in response to a number of candidate genes which are not key genes for regulating the shape of the target bacteria exceeding a preset number, returning to the step of determining the number of key protein domains based on the relationship between the number of protein domains and the error rate of the bacterial shape prediction model, and determining a new number of key protein domains;
determining new candidate genes based on the new number of key protein domains and respective influence weights of the key protein domains; and
obtaining the shape information of the target bacteria after knocking out the new candidate genes.

8. A method for identifying candidate genes which regulate a phenotype of a large number of species, comprising:
obtaining reference genome data of a large number of species and performing protein domain analysis on the reference genome data of target species;
determining a feature value dataset based on protein domains obtained from the protein domain analysis;
obtaining phenotype information of the large number of species;
training a phenotype prediction model for the large number of species based on the phenotype information and the feature value dataset, and determining influence weights of each protein domain on the phenotype of the target species according to the phenotype prediction model;
determining the candidate genes which regulate the phenotype of the target species based on the influence weights of each protein domain.

9. A method of regulating a phenotype of a large number of species, comprising:
knocking out one or more of the candidate genes of the species according to claim 8, to obtain a species with altered phenotype.

10. The method according to claim 9, wherein the species comprise a bacterium, fungus, virus, plant, or animal.

11. The method according to claim 9, wherein the phenotype comprise shape, temperature, metabolic products, height, stress resistance, or mode of locomotion.

12. A computer device comprising a memory, a processor and a computer program stored in the memory and executable on the processor, wherein the processor implements the method according to claim 1 when executing the computer program.

13. A non-transitory computer-readable storage medium storing a computer program, wherein the computer program is executed by a processor to implement the method according to claim 1.
